(19) United States
(12) Patent Application Publication
(10) Pub. No.: US 2023/0229835 A1
(43) Pub. Date: Jul. 20, 2023

(54) DYNAMIC MODEL APPLICATION BASED ON SUBJECT CHARACTERISTICS

(71) Applicant: Omniscient Neurotechnology Pty Limited, Sydney (AU)

(72) Inventors: Michael Edward Sughrue, Brighton Le Sands (AU); Stephane Philippe Doyen, Mount Kuring Gai (AU); Peter James Nicholas, South Hurstville (AU)

(21) Appl. No.: 18/096,502

(22) Filed: Jan. 12, 2023

Related U.S. Application Data
(60) Provisional application No. 63/300,573, filed on Jan. 18, 2022.

(51) Int. Cl. G06F 30/27; G16H 50/50; G16H 50/20

(52) U.S. Cl. CPC G06F 30/27; G16H 50/50; G16H 50/20; G06F 2111/10

(57) ABSTRACT

Methods, systems, and computer programs encoded on computer storage media, for selecting a model out of a number of models based on subject characteristics. One of the methods includes obtaining present subject connectivity matrix data for a present subject, obtaining present subject data describing the present subject where the present subject data is different from the present subject connectivity matrix data, determining a specific model to apply to the present subject connectivity matrix data based at least in part on the present subject data, determining the specific model using a model trained with fMRI data for brains of a plurality of past subjects and past subject data describing the past subjects, applying the specific model to identify a potential present subject brain condition based at least in part on the present subject connectivity matrix data, and taking an action based on identification of a potential present subject brain condition.

### CROSS-REFERENCE TO RELATED APPLICATION

This application claims the benefit under 35 U.S.C. § 119(e) of the filing date of U.S. Provisional Patent Application No. 63/300,573, filed Jan. 18, 2022, the entire contents of which are incorporated herein by reference.

### BACKGROUND

Diffusion tensor imaging (DTI) uses magnetic resonance images to measure diffusion of water in a human brain. The measured diffusion is used to generate images of neural tracts and corresponding white matter fibers of the subject brain. Images captured using DTI relate to the whole brain and are correspondingly complex.

Neurosurgeons typically view visual representations of DTI images for a particular purpose, for example to study operation of a certain region of the brain, study effects of certain conditions on the brain or to plan for surgery.

A region of the brain can include millions of fibers gathered as tracts. However, users (such as neurosurgeons) typically require more specific and less cluttered images in terms of operation and connections of the brain, such as identifying which tracts or fibers are connected to a region of interest. Without access to improved specificity, a neurosurgeon's study of the brain can be complex and may lead to risk in terms of identifying: 1) one or more of conditions present in the brain; 2) relevant areas for surgery; and 3) interactions between different components of the brain.

### SUMMARY

This specification describes technologies for selecting a specific model out of a number of models based on subject characteristics. These technologies generally involve directing a new subject (e.g., a brain scan of a new subject) to a specific model out of a number of models that is trained on previous subjects that are similar to the new subject. The new subject can be directed to the specific model based on subject data (e.g., age, gender, or ethnicity) or based on abstract features determined through unsupervised machine learning.

In general, one innovative aspect of the subject matter described in this specification can be embodied in methods that include the actions of: obtaining present subject connectivity matrix data for a present subject; obtaining present subject data describing the present subject where the present subject data is different from, e.g., does not include, the present subject connectivity matrix data; determining a specific model to apply to the present subject connectivity matrix data based at least in part on the present subject data, determining the specific model using a model trained with fMRI data for brains of a plurality of past subjects and past subject data describing the past subjects; applying the specific model to identify a potential present subject brain condition based at least in part on the present subject connectivity matrix data; and taking an action based at least in part on identification of a potential present subject brain condition. Other embodiments of this aspect include corresponding computer systems, apparatus, and computer programs recorded on one or more computer storage devices, each configured to perform the actions of the methods. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

The foregoing and other embodiments can each optionally include one or more of the following features, alone or in combination. In particular, one embodiment includes all the following features in combination.

Feature 1: Determining a specific model comprises using an unsupervised machine learning algorithm.

Feature 2: The unsupervised machine learning algorithm is a k-means clustering algorithm.

Feature 3: Determining a specific model comprises using a clustering method to group connectivity matrices.

Feature 4: The present subject data includes at least one of: age, gender, or ethnicity.

Feature 5: The present subject data is obtained from a DICOM header of the present subject connectivity matrix

As additional description to the embodiments described below, the present disclosure describes the following embodiments.

Embodiment 1 is a method, comprising: obtaining present subject connectivity matrix data for a present subject; obtaining present subject data describing the present subject where the present subject data is different from the present subject connectivity matrix data; determining a specific model to apply to the present subject connectivity matrix data based at least in part on the present subject data, determining the specific model using a model trained with fMRI data for brains of a plurality of past subjects and past subject data describing the past subjects; applying the specific model to identify a potential present subject brain condition based at least in part on the present subject connectivity matrix data; and taking an action based at least in part on identification of a potential present subject brain condition.
Embodiment 2 is the method of embodiment 1, wherein determining a specific model comprises using an unsupervised machine learning algorithm.
Embodiment 3 is the method of embodiments 1 or 2, wherein the unsupervised machine learning algorithm is a k-means clustering algorithm.
Embodiment 4 is the method of any one of embodiments 1 through 3, wherein determining a specific model comprises using a clustering method to group connectivity matrices.
Embodiment 5 is the method of any one of embodiments 1 through 4, wherein the present subject data includes at least one of: age, gender, or ethnicity.
Embodiment 6 is the method of embodiment 1, wherein the present subject data is obtained from a DICOM header of the present subject connectivity matrix.
Embodiment 7 is directed to one or more computer-readable devices having instructions stored thereon, that when executed by one or more processors, cause the performance of actions according to the method of any one of embodiments 1 through 6.
Embodiment 8 is directed to a system comprising one or more computers and one or more storage devices on which are stored instructions that are operable, when executed by the one or more computers, to cause the one or more computers to perform operations according to the method of any one of embodiments 1 through 6.

The subject matter described in this specification can be implemented in particular embodiments so as to realize one or more of the following advantages. When data is regrouped into specific groups (e.g., by age, gender, or ethnicity) then models can be trained specifically for those groups, and the resulting models can be more accurate on their respective groups than a generalized model.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIGS. 1A and 1B form a schematic block diagram of a computer system upon which arrangements described can be practiced;

FIG. 2A shows a method of segmenting datasets and training models.

FIG. 2B shows another method of segmenting datasets and training models.

FIG. 3A shows a method of determining and applying a specific model.

FIG. 3B shows another method of determining and applying a specific model.

FIG. 4 is a flowchart of a method of applying a specific model to obtained data.

Like reference numbers and designations in the various drawings indicate like elements.

### DETAILED DESCRIPTION

Where reference is made in any one or more of the accompanying drawings to steps and/or features, which have the same reference numerals, those steps and/or features have for the purposes of this description the same function(s) or operation(s), unless the contrary intention appears.

A brain atlas is a representation of the brain. More specifically, a brain atlas can be serial sections along different anatomical planes of the healthy or diseased developing or adult animal or human brain where each relevant brain structure is assigned a number of coordinates to define its outline or volume. A brain atlas can be a result from visual brain mapping and may include anatomical, cytological, genetic and/or functional features. A functional brain atlas can be made up of a specified number of regions of interest, where the regions are typically defined as spatially contiguous and functionally coherent patches of gray matter. One can refer to the identified sections/regions of the brain as parcellations of the brain. For example, one can delineate 180 regions/parcellations per hemisphere where the regions/parcellations are bounded by sharp changes in cortical architecture, function, connectivity, and/or topography. Such parcellations can be determined based on a precisely aligned group (e.g., more than 200) healthy young adults. More generally, the number of parcellations making up a brain can be more than 50, more than 100, more than 250, more than 350, more than 500, or more than 1000. The volume of the parcellations can range (or be the same) where no individual parcellation is smaller than a cubic millimeter, smaller than 50 cubic millimeters, smaller than 100 cubic millimeters, or smaller than 500 cubic millimeters and/or where no individual parcellation is larger than ¼ of a brain hemisphere, larger than a ⅙ of a brain hemisphere, larger than a 1/12 of a brain hemisphere, or larger than 2 cubic centimeters. For example, parcellations can range in size from 50 cubic millimeters to 2 cubic centimeters and range in number between 100 and 400. Such parcellations do not need to be (but can be) uniform in volume and/or shape.

The arrangements described allow a user of a medical image display system, such as a neurosurgeon, to view DTI image data in a manner that just shows specified network(s) or interconnections of particular tracts and fibers corresponding to a particular function or structure of the brain. A graphical representation that identifies particular parcellations and corresponding tracts, or portions of tracts, relevant to the structure can be provided. A network of the brain can be constructed based upon parcellations of the brain and corresponding structural and functional connections.

The arrangements described allow use of DTI images for a subject to be provided in an improved manner so that a user can identify individual tracts or fibers relevant to interconnected or inter-operational portions of the brain. For example, tracts (or fibers) associated with particular parcellations or other known anatomical structures of the brain and the spatial relationships of the tracts (or fibers) with the parcellation can be represented graphically. Compared to previous solutions where all tracts in a region would be represented, thereby occluding relationships between tracts (or fibers) with one another and with certain portions of the brain, the user/viewer obtains more specific, e.g., more specific and more granular, image data and a more clinically meaningful image. A neurosurgeon, for example, is thereby allowed an improved study of a subject brain, for example interconnections of particular tracts, regions, and networks. Given the more clinically meaningful image, the neurosurgeon can better understand connections and operations of the subject brain. Decisions relating to conditions, operation of the subject brain and procedures to be performed on the subject brain can be improved, thereby increasing patient safety and standard of care.

In order to allow a representation of the image data that isolates and identifies interconnections associated with a grouping, function or region of the brain, this specification provides a model mapping elements of the brain using atlas parcellations in accordance with a three-dimensional model of a brain. U.S. Pat. Nos. 11,055,849 and 11,145,119, both incorporated herein by reference in their entirety, describe the creation of a three-dimensional model of a brain and differential brain network analysis. The model is effectively a library of neuro-anatomy that can be used to assign parcellations of the brain into networks for particular function(s). Implementations of a system described in this specification can use the structure of the model to determine corresponding data from a DTI image and use that DTI data to graphically represent a particular network of the brain. Such a library structure further allows a user such as a neurosurgeon to use the graphical user interface accurately and intuitively to obtain a visual reconstruction of the brain of a particular subject to view network interconnections.

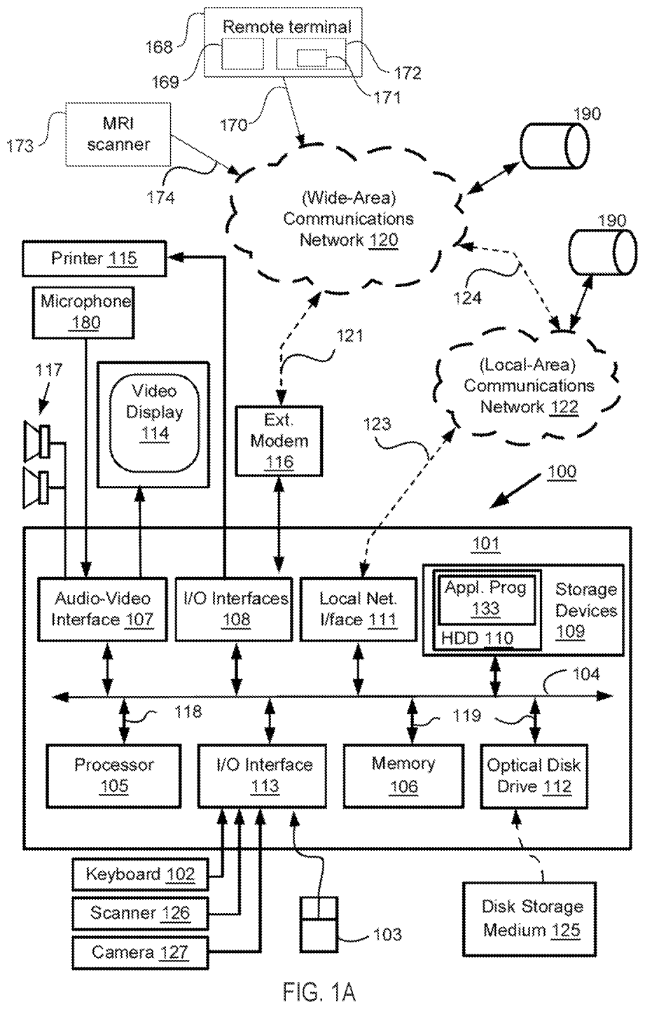

A computing device can perform the arrangements described. FIGS. 1A and 1B depict a computer system 100, upon which one can practice the various arrangements described.

As seen in FIG. 1A, the computer system 100 includes: a computer module 101; input devices such as a keyboard 102, a mouse pointer device 103, a scanner 126, a camera 127, and a microphone 180; and output devices including a printer 115, a display device 114 and loudspeakers 117. An external Modulator-Demodulator (Modem) transceiver device 116 may be used by the computer module 101 for communicating to and from a communications network 120 via a connection 121. The communications network 120 may be a wide-area network (WAN), such as the Internet, a cellular telecommunications network, or a private WAN. Where the connection 121 is a telephone line, the modem 116 may be a traditional “dial-up” modem. Alternatively, where the connection 121 is a high capacity (e.g., cable) connection, the modem 116 may be a broadband modem. A wireless modem may also be used for wireless connection to the communications network 120.

The computer module 101 typically includes at least one processor unit 105, and a memory unit 106. For example, the memory unit 106 may have semiconductor random access memory (RAM) and semiconductor read only memory (ROM). The computer module 101 also includes an number of input/output (I/O) interfaces including: an audio-video interface 107 that couples to the video display 114, loudspeakers 117 and microphone 180; an I/O interface 113 that couples to the keyboard 102, mouse 103, scanner 126, camera 127 and optionally a joystick or other human interface device (not illustrated); and an interface 108 for the external modem 116 and printer 115. In some implementations, the modem 116 may be incorporated within the computer module 101, for example within the interface 108. The computer module 101 also has a local network interface 111, which permits coupling of the computer system 100 via a connection 123 to a local-area communications network 122, known as a Local Area Network (LAN). As illustrated in FIG. 1A, the local communications network 122 may also couple to the wide network 120 via a connection 124, which would typically include a so-called “firewall” device or device of similar functionality. The local network interface 111 may comprise an Ethernet circuit card, a Bluetooth® wireless arrangement or an IEEE 802.11 wireless arrangement; however, numerous other types of interfaces may be practiced for the interface 111.

The module 101 can be connected with an image capture device 197 via the network 120. The device 197 can capture images of a subject brain using each of diffusor tension imaging and magnetic resonance imaging (MM) techniques. The captured images are typically in standard formats such as DICOM format and OpenfMRI format respectively. The module 101 can receive DTI and MRI images the device 197 via the network 120. Alternatively, the DTI and MM images can be received by the module 101 from a remote server, such as a cloud server 199, via the network 120. In other arrangements, the module 101 may be an integral part of one of the image capture device 197 and the server 199.

The I/O interfaces 108 and 113 may afford either or both of serial and parallel connectivity, the former typically being implemented according to the Universal Serial Bus (USB) standards and having corresponding USB connectors (not illustrated). Storage devices 109 are provided and typically include a hard disk drive (HDD) 110. Other storage devices such as a floppy disk drive and a magnetic tape drive (not illustrated) may also be used. An optical disk drive 112 is typically provided to act as a non-volatile source of data. Portable memory devices, such optical disks (e.g., CD-ROM, DVD, Blu-ray™ Disc), USB-RAM, portable, external hard drives, and floppy disks, for example, may be used as appropriate sources of data to the system 100.

The components 105 to 113 of the computer module 101 typically communicate via an interconnected bus 104 and in a manner that results in a conventional mode of operation of the computer system 100 known to those in the relevant art. For example, the processor 105 is coupled to the system bus 104 using a connection 118. Likewise, the memory 106 and optical disk drive 112 are coupled to the system bus 104 by connections 119. Examples of computers on which the described arrangements can be practiced include IBM-PC's and compatibles, Sun Sparcstations, Apple Mac™ or like computer systems.

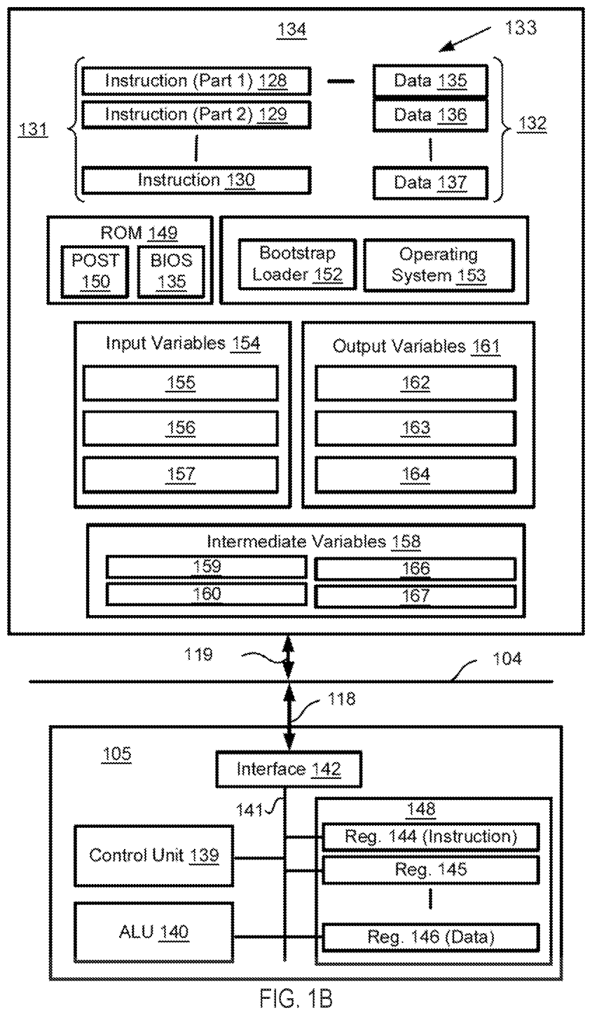

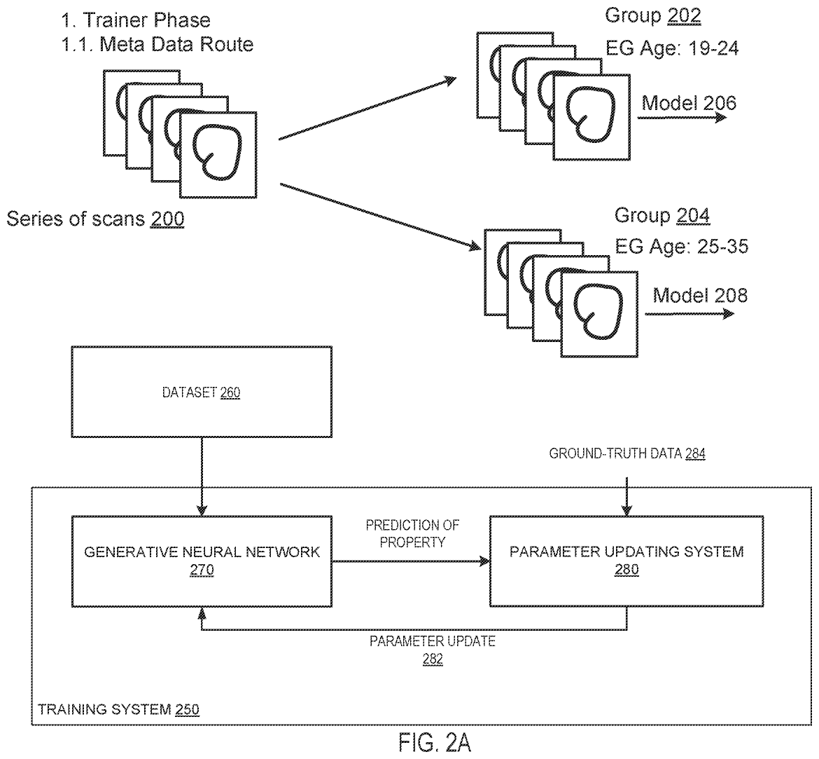

The method described may be implemented using the computer system 100 wherein the processes of FIGS. 2-4, to be described, may be implemented as one or more software application programs 133 executable within the computer system 100. In particular, the steps of the method described are effected by instructions 131 (see FIG. 1B) in the software 133 that are carried out within the computer system 100. The software instructions 131 may be formed as one or more code modules, each for performing one or more particular tasks. The software may also be divided into two separate parts, in which a first part and the corresponding code modules performs the described methods and a second part and the corresponding code modules manage a user interface between the first part and the user.

The software may be stored in a computer readable medium, including the storage devices described below, for example. The software is loaded into the computer system 100 from the computer readable medium, and then executed by the computer system 100. A computer readable medium having such software or computer program recorded on the computer readable medium is a computer program product. The use of the computer program product in the computer system 100 preferably effects an advantageous apparatus for providing a display of a neurological image.

The software 133 is typically stored in the HDD 110 or the memory 106. The software is loaded into the computer system 100 from a computer readable medium, and executed by the computer system 100. Thus, for example, the software 133 may be stored on an optically readable disk storage medium (e.g., CD-ROM) 125 that is read by the optical disk drive 112. A computer readable medium having such software or computer program recorded on it is a computer program product. The use of the computer program product in the computer system 100 preferably effects an apparatus for providing a display of a neurological image.

In some instances, the application programs 133 may be supplied to the user encoded on one or more CD-ROMs 125 and read via the corresponding drive 112, or alternatively may be read by the user from the networks 120 or 122. Still further, the software can also be loaded into the computer system 100 from other computer readable media. Computer readable storage media refers to any non-transitory tangible storage medium that provides recorded instructions and/or data to the computer system 100 for execution and/or processing. Examples of such storage media include floppy disks, magnetic tape, CD-ROM, DVD, Blu-ray™ Disc, a hard disk drive, a ROM or integrated circuit, USB memory, a magneto-optical disk, or a computer readable card such as a PCMCIA card and the like, whether or not such devices are internal or external of the computer module 101. Examples of transitory or non-tangible computer readable transmission media that may also participate in the provision of software, application programs, instructions and/or data to the computer module 101 include radio or infra-red transmission channels as well as a network connection to another computer or networked device, and the Internet or Intranets including e-mail transmissions and information recorded on Websites and the like.

The second part of the application programs 133 and the corresponding code modules mentioned above may be executed to implement one or more graphical user interfaces (GUIs) to be rendered or otherwise represented upon the display 114. Through manipulation of typically the keyboard 102 and the mouse 103, a user of the computer system 100 and the application may manipulate the interface in a functionally adaptable manner to provide controlling commands and/or input to the applications associated with the GUI(s). Other forms of functionally adaptable user interfaces may also be implemented, such as an audio interface utilizing speech prompts output via the loudspeakers 117 and user voice commands input via the microphone 180.

FIG. 1B is a detailed schematic block diagram of the processor 105 and a “memory” 134. The memory 134 represents a logical aggregation of all the memory modules (including the HDD 109 and semiconductor memory 106) that can be accessed by the computer module 101 in FIG. 1A.

When the computer module 101 is initially powered up, a power-on self-test (POST) program 150 executes. The POST program 150 is typically stored in a ROM 149 of the semiconductor memory 106 of FIG. 1A. A hardware device such as the ROM 149 storing software is sometimes referred to as firmware. The POST program 150 examines hardware within the computer module 101 to ensure proper functioning and typically checks the processor 105, the memory 134 (109, 106), and a basic input-output systems software (BIOS) module 151, also typically stored in the ROM 149, for correct operation. Once the POST program 150 has run successfully, the BIOS 151 activates the hard disk drive 110 of FIG. 1A. Activation of the hard disk drive 110 causes a bootstrap loader program 152 that is resident on the hard disk drive 110 to execute via the processor 105. This loads an operating system 153 into the RAM memory 106, upon which the operating system 153 commences operation. The operating system 153 is a system level application, executable by the processor 105, to fulfil various high level functions, including processor management, memory management, device management, storage management, software application interface, and generic user interface.

The operating system 153 manages the memory 134 (109, 106) to ensure that each process or application running on the computer module 101 has sufficient memory in which to execute without colliding with memory allocated to another process. Furthermore, the different types of memory available in the system 100 of FIG. 1A must be used properly so that each process can run effectively. Accordingly, the aggregated memory 134 is not intended to illustrate how particular segments of memory are allocated (unless otherwise stated), but rather to provide a general view of the memory accessible by the computer system 100 and how such is used.

As shown in FIG. 1B, the processor 105 includes a number of functional modules including a control unit 139, an arithmetic logic unit (ALU) 140, and a local or internal memory 148, sometimes called a cache memory. The cache memory 148 typically includes a number of storage registers 144-146 in a register section. One or more internal busses 141 functionally interconnect these functional modules. The processor 105 typically also has one or more interfaces 142 for communicating with external devices via the system bus 104, using a connection 118. The memory 134 is coupled to the bus 104 using a connection 119.

The application program 133 includes a sequence of instructions 131 that may include conditional branch and loop instructions. The program 133 may also include data 132 which is used in execution of the program 133. The instructions 131 and the data 132 are stored in memory locations 128, 129, 130 and 135, 136, 137, respectively. Depending upon the relative size of the instructions 131 and the memory locations 128-130, a particular instruction may be stored in a single memory location as depicted by the instruction shown in the memory location 130. Alternately, an instruction may be segmented into a number of parts each of which is stored in a separate memory location, as depicted by the instruction segments shown in the memory locations 128 and 129.

In general, the processor 105 is given a set of instructions which are executed therein. The processor 105 waits for a subsequent input, to which the processor 105 reacts to by executing another set of instructions. Each input may be provided from one or more of a number of sources, including data generated by one or more of the input devices 102, 103, data received from an external source across one of the networks 120, 102, data retrieved from one of the storage devices 106, 109 or data retrieved from a storage medium 125 inserted into the corresponding reader 112, all depicted in FIG. 1A. The execution of a set of the instructions may in some cases result in output of data. Execution may also involve storing data or variables to the memory 134.

The described arrangements use input variables 154, which are stored in the memory 134 in corresponding memory locations 155, 156, 157. The described arrangements produce output variables 161, which are stored in the memory 134 in corresponding memory locations 162, 163, 164. Intermediate variables 158 may be stored in memory locations 159, 160, 166 and 167.

Referring to the processor 105 of FIG. 1B, the registers 144, 145, 146, the arithmetic logic unit (ALU) 140, and the control unit 139 work together to perform sequences of micro-operations needed to perform “fetch, decode, and execute” cycles for every instruction in the instruction set making up the program 133. Each fetch, decode, and execute cycle comprises:

a fetch operation, which fetches or reads an instruction 131 from a memory location 128, 129, 130;

a decode operation in which the control unit 139 determines which instruction has been fetched; and

an execute operation in which the control unit 139 and/or the ALU 140 execute the instruction.

Thereafter, a further fetch, decode, and execute cycle for the next instruction may be executed. Similarly, a store cycle may be performed by which the control unit 139 stores or writes a value to a memory location 132.

Each step or sub-process in the processes of FIGS. 2-4 is associated with one or more segments of the program 133 and is performed by the register section 144, 145, 147, the ALU 140, and the control unit 139 in the processor 105 working together to perform the fetch, decode, and execute cycles for every instruction in the instruction set for the noted segments of the program 133.

It can be useful to regroup data to create more accurate models for specific groups. For example, a brain atlas may be different for, e.g., a seven year old or a sixty year old. For example, cognitive functions can decline with age. Connectivity patterns which might be normal at a certain age (e.g., a subject at the age of 90 having poor memory) are not normal at other ages (e.g., a subject at the age of 25 having poor memory). Establishing subtypes and groups can put these cases in separate categories that are more homogenous, which can provide less variance when training models. Similarly, parcellations can differ between different groups. Some examples of groups that can be analyzed separately are: ethnicity, gender, age, or mental conditions (e.g., categories for the Diagnostic and Statistical Manual of Mental Disorders (DSM-5)). These and other groups can often be found on new scans, e.g., on a DICOM header. For this reason, it can be advantageous to create different models for analyzing these groups. An exemplary method for regrouping data is shown in FIGS. 2A and 2B.

FIG. 2A illustrates a method of training a model based on patient subject data. For example, a series of previous connectivity matrices can be used to train a model that can predict whether a specific patient has a particular condition. Training a model based on subject data can be useful because the distinctions between groups are quantitatively or qualitatively describable by a user, and a scan can be directed to its respective group based on these distinctions. A series of connectivity matrices with a property (e.g., patient has a specified condition) and without the property can be selected to train a model. If the model is trained without being regrouped into specific subgroups, it can be less accurate. However, if the data is regrouped into specific groups (e.g., by age) then the resulting models can be more accurate on their respective groups. For example, area under a curve (AUC) is a common performance metric. In some embodiments, a model that is not regrouped (i.e., is trained on all data together) can have an AUC of 0.65 for predicting whether a given connectivity matrix exhibits a symptom or not. Meanwhile, a model that is regrouped by age (i.e., the model is trained only on a specific age group) can have an AUC of 0.75 for predicting whether a given connectivity matrix within its respective age group exhibits a symptom or not. In the example illustrated in FIG. 2A, the series of connectivity matrices 200 are sorted based on patient subject data, specifically age group. As illustrated, the series of connectivity matrices 202 are sorted into two age groups 204 and 206. Group 204 contains connectivity matrices of patients that are between 19 and 24 years of age, and group 206 contains connectivity matrices of patients that are between 25 and 35 years of age. Each group 204, 206 can independently serve as a dataset to train a model that can predict the property (e.g., a specified condition). In some implementations, the data can be segmented multiple times into more groups. For example, the data can be segmented by gender, then segmented by age bracket, and then segmented by mental condition. The resulting segmented data can be used to analyze similar functional connectivity mappings. For example, a female seven year old may not have the same functional connectivity mappings as a male sixty year old, and therefore would not be placed in the same group when the data is segmented and regrouped. Instead, the brain of the female seven year old would be compared to brains of other females of about the same age.

Thus, a model can then be trained for each respective group using a machine learning algorithm (e.g., linear regression models, support vector machines (SVM), neural networks, decision trees) to predict whether the property is present. For example, model 206 can be trained using only the connectivity matrices belonging to group 202, and model 208 can be trained using only the connectivity matrices belonging to group 204. The machine learning algorithm can be a supervised machine learning algorithm or an unsupervised machine learning algorithm. As an example, the training system 250 is an exemplary algorithm that can train a model given a dataset of connectivity matrices (e.g., group 204 or group 206). As illustrated, a dataset 260 is input into a generative neural network 270. The generative neural network 270 analyzes a connectivity matrix of the dataset 260 and predicts whether the property is reflected in the connectivity matrix. Ground-truth data 284 is used in the parameter updating system 280 to determine whether the generative neural network 270 correctly predicts presence of the property. Feedback regarding the predictions of the generative neural network 270 compared to the ground truth data is returned as a parameter update 282. This training system 250 is repeated until the generative neural network can correctly make predictions, e.g., until the generative neural network can correctly predict the presence of the property for an acceptable percentage of the connectivity matrices (e.g., 99% of the connectivity matrices). This training system 250 is one exemplary method to train a model, but other methods of training (e.g., decision trees, linear regression, SVM, etc.) can be used to train a model to predict the presence of a property in an individual connectivity matrix.

FIG. 2B illustrates a second method of training a model based on patient data. However, this method does not utilize patient subject data and relies solely on the connectivity matrices of the patient. For example, any information contained in a DICOM header (e.g., age, gender, and ethnicity) is not necessary for this method, and the method can rely solely on the connectivity matrix. However, in some implementations, subject data separate from the connectivity data, e.g., information contained in the DICOM header, can also be utilized.

In this method, a series of connectivity matrices 220 with a property (e.g., a symptom) and without the property can be selected to train models. The series of connectivity matrices 220 are sorted into separate groups using a supervised or unsupervised machine learning algorithm. For example, the series of connectivity matrices 220 can be sorted into separate groups 222, 224, 226 using a clustering technique (e.g., agglomerative clustering, k-means clustering, mean-shift clustering, Density-Based Spatial Clustering of Applications with Noise (DBSCAN), Gaussian Mixture Models (GMM), or hierarchical clustering). An advantage of using a clustering technique to separate the series of connectivity matrices 220 is that the clusters are not fixed and can determine connectivity matrices that are more similar to each other than for example simply grouping the connectivity matrices of subjects by the subject's age. In other words, the clusters can be based on properties that are not easily distinguishable, and the differences between the clusters can be more difficult to distinguish and comprehend in human terms. For example, small differences in the connectivity matrices that are not easily distinguished by humans may be significant in terms of predictions of a specified condition. The matrices can be vectorized according to standard techniques (e.g., by stacking all the rows of the upper triangle of a symmetric matric to create a long vector) and then the clustering technique, e.g., an agglomerative clustering technique or a k-means clustering technique, can be applied to a set of resulting vectorized matrices. Prototyping can typically be accomplished with at least 200 examples. A model can typically be trained with at least 2000 examples including both matrices for patients with the property and matrices without the property.

Once the series of connectivity matrices 220 is separated into groups 222, 224, 226, a classifier 228 can be trained to predict to which group 222, 224, 226 an individual connectivity matrix belongs. For example, a classifier can be trained using a machine learning algorithm (e.g. neural networks, decision trees, linear regression, SVM, etc.) to predict to which group an individual connectivity matrix belongs. The classifier 228 that predicts which group an individual connectivity matrix belongs can be also trained using a training system similar to training system 250.

After the classifier 228 is trained and can identify to which group an individual connectivity matrix for a specified subject belongs, individual models for predicting the property can be trained for each respective group 222, 224, 226. For example, model 230 can be trained using only the connectivity matrices belonging to group 222, model 232 can be trained using only the connectivity matrices belonging to group 224, and model 234 can be trained using only the connectivity matrices belonging to group 226. The individual model for each respective group 222, 224, 226 will be more accurate than a general model for the entire series of connectivity matrices 220 because although the groups 222, 224, 226 are not distinguishable in human terms (e.g., age, gender, etc.) the connectivity matrices in each group can be more similar to other connectivity matrices in the same group, e.g., because small differences in the connectivity matrices that are not easily distinguished by humans may result in significantly different predictions.

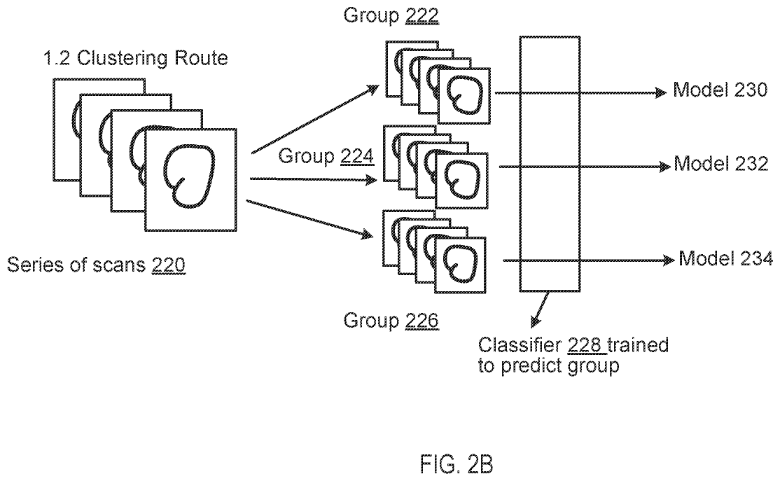

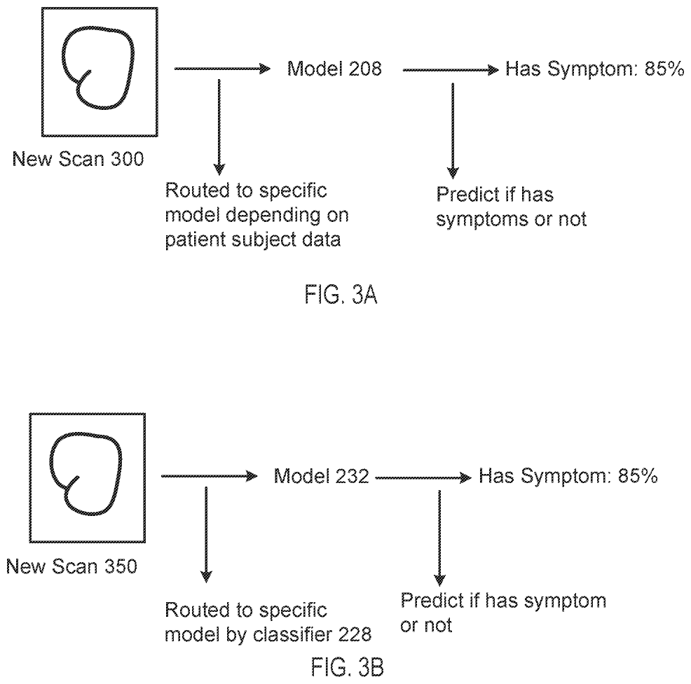

FIG. 3A illustrates a method of predicting a property (e.g., a symptom) in a new connectivity matrix 300 by applying a model that is trained by segmenting and regrouping the training data. For example, FIG. 3A illustrates a method of predicting a property with a model that has been trained via the method of FIG. 2A (i.e., the models have been trained with groups that are separated by subject data). In this case, the new connectivity matrix 300 can be sorted into its respective group by analyzing the subject data (e.g., ethnicity, age, gender, etc.). Based on the group that the new connectivity matrix 300 is sorted into, a particular model can be selected that was trained on that group. For example, if the new connectivity matrix 300 was sorted into group 204 (e.g., age group 25-35), then model 208 can be selected. Because model 208 was trained with group 204, model 208 will be most accurate on connectivity matrices belonging to group 204. The selected model 208 can then predict whether the property (e.g., symptom) is present in the new connectivity matrix 300. In FIG. 3A, the model predicts with 85% certainty that the property is present in the new connectivity matrix 300 based on the training from FIG. 2A.

FIG. 3B illustrates another method of predicting a property (e.g., a symptom) in a new connectivity matrix 350 by applying a model that is trained by segmenting and regrouping the training data. For example, FIG. 3B illustrates a method of predicting a property with a model that has been trained via the method of FIG. 2B. In this case, a human cannot necessarily distinguish to which group a new connectivity matrix 350 belongs. Therefore, the new connectivity matrix 350 needs to be classified by the classifier 228 trained to predict to which group the new connectivity matrix 350 belongs and therefore which model is most accurate for the new scan 350. For example, if the new connectivity matrix 350 was determined to belong to group 224, then model 232 can be selected. Because model 232 was trained with group 224, model 232 will be most accurate on connectivity matrices belonging to group 224. The selected model 232 can then predict whether the property (e.g., condition) is present in the new connectivity matrix 350. In the present example, the model predicts with 85% certainty that the property is present in the new connectivity matrix 350.

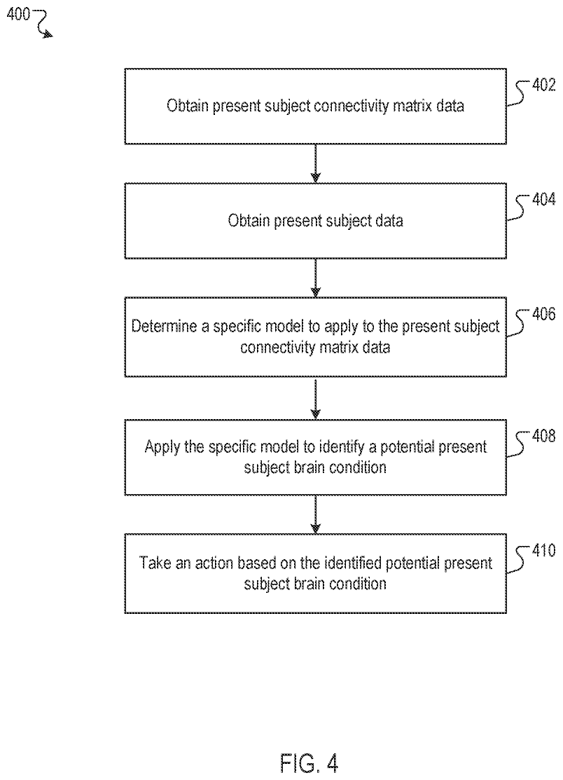

FIG. 4 illustrates a flowchart of a method 400 of obtaining data and applying a specific model to identify a potential brain condition. First, connectivity matrix data of a present subject (e.g., a patient) is obtained 402. For example, connectivity matrix data can be obtained by capturing images of a subject brain using each of diffusor tension imaging and magnetic resonance imaging (MM) techniques. The captured images are typically in standard formats such as DICOM format and OpenfMRI format respectively. Connectivity matrix data can also be obtained by capturing DTI and MM images. Alternatively, the DTI and MRI images can be obtained from a remote server, such as a cloud server, via a network.

After obtaining 402 connectivity matrix data for a present subject, the method 400 includes obtaining 404 present subject data. The present subject data can be data about the present subject other than the connectivity matrix data. For example, present subject data can be data about the present subject (e.g., gender, age, and mental conditions), e.g., data pulled from the DICOM header.

After obtaining 404 present subject data, the method 400 includes determining a specific model to apply to the present subject connectivity matrix data. Determining a specific model to apply can be similar to FIG. 3A, in which the present subject data is used to select which model should be run on a new scan. For example, present subject data (e.g., pulled from a DICOM header) can be utilized to select a model that best fits the connectivity matrix data. In other embodiments of the method 400, determining the specific model to apply can be similar to FIG. 3B, in which present subject data is not used to select which model should be run on the new scan. For example, a classifier that was trained on clustered data can be used to determine which model should be run on the new scan. In other implementations, determining a specific model to apply can include portions of the methods of FIGS. 3A and 3B (e.g., present subject data can be used to determine a specific model, and a classifier can be used to determine a specific model).

After determining 406 a specific model to apply, the method 400 includes applying 408 the specific model to identify a potential present subject brain condition. For example, the present subject brain condition can be a category of the DSM-5 or RDoc.

After applying 408 the specific model, the method 400 can include taking an action 410 based on the identified potential present subject brain condition. For example, taking an action can include providing a value for certainty of the model. For example, the specific model can provide a value as a percentage of certainty (e.g., 85% certainty) that the connectivity matrix data has or does not have the potential brain condition.

The arrangements described are applicable to the medical image capture and data processing industries and particularly for the medical industries related to neurology and associated healthcare.

The foregoing describes only some embodiments of the present invention, and modifications and/or changes can be made thereto without departing from the scope and spirit of the invention, the embodiments being illustrative and not restrictive.

In the context of this specification, the word “comprising” means “including principally but not necessarily solely” or “having” or “including”, and not “consisting only of”. Variations of the word “comprising”, such as “comprise” and “comprises” have correspondingly varied meanings.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone, running a messaging application, and receiving responsive messages from the user in return.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method comprising:
obtaining present subject connectivity matrix data for a present subject;
obtaining present subject data describing the present subject where the present subject data is different from the present subject connectivity matrix data;
determining a specific model to apply to the present subject connectivity matrix data based at least in part on the present subject data, determining the specific model using a model trained with fMRI data for brains of a plurality of past subjects and past subject data describing the plurality of past subjects;
applying the specific model to identify a potential present subject brain condition based at least in part on the present subject connectivity matrix data; and
taking an action based at least in part on identification of a potential present subject brain condition.

2. The method of claim 1, wherein determining a specific model comprises using an unsupervised machine learning algorithm.

3. The method of claim 2, wherein the unsupervised machine learning algorithm is a k-means clustering algorithm.

4. The method of claim 1, wherein determining a specific model comprises using a clustering method to group connectivity matrices.

5. The method of claim 1, wherein the present subject data includes at least one of: age, gender, or ethnicity.

6. The method of claim 5, wherein the present subject data is obtained from a DICOM header of the present subject connectivity matrix.

7. A computer program product, tangibly embodied in a machine readable storage device, the computer program product being operable to cause a data processing apparatus to:
obtain present subject connectivity matrix data for a present subject;
obtain present subject data describing the present subject where the present subject data is different from the present subject connectivity matrix data;
determine a specific model to apply to the present subject connectivity matrix data based at least in part on the present subject data, determine the specific model using a model trained with fMRI data for brains of a plurality of past subjects and past subject data describing the past subjects;
apply the specific model to identify a potential present subject brain condition based at least in part on the present subject connectivity matrix data; and
take an action based at least in part on identification of a potential present subject brain condition.

8. The computer program product of claim 7, wherein to determine a specific model the computer program product is operable to cause a data processing apparatus to run an unsupervised machine learning algorithm.

9. The computer program product of claim 8, wherein the unsupervised machine learning algorithm is a k-means clustering algorithm.

10. The computer program product of claim 7, wherein to determine a specific model the computer program product is operable to cause a data processing apparatus to run a clustering method to group connectivity matrices.

11. The computer program product of claim 7, wherein the present subject data includes at least one of: age, gender, or ethnicity.

12. The computer program product of claim 7, wherein to obtain the present subject data the computer program product is operable to cause a data processing apparatus to obtain the present subject data from a DICOM header of the present subject connectivity matrix.

13. A system comprising:
one or more computers and one or more storage devices on which are stored instructions that are operable, when executed by the one or more computers, to cause the one or more computers to perform operations comprising:
obtaining present subject connectivity matrix data for a present subject;
obtaining present subject data describing the present subject where the present subject data is different from the present subject connectivity matrix data;
determining a specific model to apply to the present subject connectivity matrix data based at least in part on the present subject data, determining the specific model using a model trained with fMRI data for brains of a plurality of past subjects and past subject data describing the past subjects;
applying the specific model to identify a potential present subject brain condition based at least in part on the present subject connectivity matrix data; and
taking an action based at least in part on identification of a potential present subject brain condition.

14. The system of claim 13, wherein to determine a specific model the stored instructions are operable to cause a data processing apparatus to run an unsupervised machine learning method.

15. The system of claim 14, wherein method is a k-means clustering algorithm.

16. The system of claim 13, wherein to determine a specific model the stored instructions are operable to cause a data processing apparatus to run a clustering method to group connectivity matrices.

17. The system of claim 13, wherein the present subject data includes at least one of: age, gender, or ethnicity.

18. The system of claim 13, wherein to obtain the present subject data the stored instructions are operable to cause a data processing apparatus to obtain the present subject data from a DICOM header of the present subject connectivity matrix.
